SEQUENCE LISTING (12 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 154 aa, Macaca mulatta
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRDTKDLISNINVIVLELKGSETTLMCEYADETATIVEFLNRWITFCQSIISTLT

SEQ ID NO: 3 - protein, 154 aa, Macaca fascicularis
MYRMQLLSCIALSLALVTNSAPTSSSTKKTQLQLEHLLLDLQMILNGINNYKNPKLTRMLTFKFYMPKKATELRHLQCLEEELKPLEEVLNLAQSKSFHLRDTKDLISNINVIVLELKGSETTLMCEYADETATIVEFLNRWITFCQSIISTLT

SEQ ID NO: 4 - protein, 154 aa, Cercocebus torquatus
MYRMQLLSCIALSLALVTNSAPTSRSTKKTQLQLEHLLLDLQMILNGINNYKNPKLTRMLTFKFYMPKKATELKHLQCLEEELKPLEEVLNLAQSKNFHLRDTKDLISNINVIVLELKGSETTLMCEYADETATIVEFLNRWITFCQSIISTLT

SEQ ID NO: 5 - protein, 724 aa, Artificial Sequence
  Human serum albumin-IL2 fusion protein with mutations of the invention
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPTSSSTAATQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 330 aa, Homo sapiens [sequence omitted]

SEQ ID NO: 8 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - DNA, 40 bp, Artificial Sequence
  Synthetic oligonucleotide
ccgggtgccgccccaacttcaagtagtactgccgccacag

SEQ ID NO: 10 - DNA, 38 bp, Artificial Sequence
  Synthetic Oligonucleotide
ctgtgtggcggcagtactacttgaagttggggcggcac

SEQ ID NO: 11 - DNA, 989 bp, Artificial Sequence
  DNA sequence encoding mature light chain of KS-IL-2 (K8A K9A)
gagatcgtgctgacccagtcccccgccaccctgtccctgtcccccggcgagcgcgtgaccctgacctgctccgcctcctcctccgtgtcctacatgctgtggtaccagcagaagccaggatcctcgcccaaaccctggatttttgacacatccaacctggcttctggattccctgctcgcttcagtggcagtgggtctgggacctcttactctctcataatcagcagcatggaggctgaagatgctgccacttattactgccatcagcggagtggttacccgtacacgttcggaggggggaccaagctggaaataaaacgtaagatcccgcaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - DNA, 2576 bp, Artificial Sequence
  DNA sequence encoding mature heavy chain-IL2 fusion moiety of KS-IL2 (K8A K9A)
cagatccagttggtgcagtctggagctgaggtgaagaagcctggagagacagtcaagatctcctgcaaggcttctgggtataccttcacaaactatggaatgaactgggtgaagcagactccaggaaagggtttaaagtggatgggctggataaacacctacactggagaaccaacatatgctgatgacttcaagggacggtttgccttctctttggaaacctctaccagcactgcctttttgcagatcaacaatctcagaagtgaggacacggctacatatttctgtgtaagatttatttctaagggggactactggggtcaaggaacgtcagtcaccgtctcctcaggtaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagccccaacttcaagtagtactgccgccacacagctgcaactggagcatctcctgctggatctccagatgattctgaatggaattaacaactacaagaatcccaaactcaccaggatgctcacattcaagttctacatgcccaagaaggccacagagctcaaacatctccagtgtctagaggaggaactcaaacctctggaggaagtgctaaacctcgctcagagcaaaaacttccacttaagacctagggacttaatcagcaatatcaacgtaatagttctggaactaaagggatccgaaacaacattcatgtgtgaatatgctgatgagacagcaaccattgtagaattcctaaacagatggattaccttttgtcaaagcatcatctcaacactaacttgataattaagtgctcgag

(19) United States
(12) Patent Application Publication
(10) Pub. No.: US 2005/0069521 A1
(43) Pub. Date: Mar. 31, 2005

(54) Enhancing the circulating half-life of interleukin-2 proteins

(75) Inventors: Stephen D. Gillies, Carlisle, MA (US); Scott Lauder, Boxborough, MA (US); Jeffrey Way, Cambridge, MA (US)

Correspondence Address: TESTA, HURWITZ & THIBEAULT, LLP, HIGH STREET TOWER, 125 HIGH STREET, BOSTON, MA, 02110, US

(73) Assignee: EMD Lexigen Research Center Corp., Billerica, MA (US)

(21) Appl. No.: 10/928,305

(22) Filed: Aug. 27, 2004

Related U.S. Application Data
(60) Provisional application No. 60/498,618, filed on Aug. 28, 2003.

(51) Int. Cl. A61K 38/20; C07K 14/54; C07H 21/04; C12P 21/04; A61K 39/00

(52) U.S. Cl. 424/85.2; 530/351; 424/192.1; 435/69.52; 435/320.1; 435/325; 536/23.5

(57) ABSTRACT

Disclosed are compositions and methods for enhancing the circulating half-life of interleukin-2 proteins.

### REFERENCE TO RELATED APPLICATIONS

This application claims the benefit of U.S. Ser. No. 60/498,618, filed Aug. 28, 2003, the content of which is incorporated by reference.

### FIELD OF THE INVENTION

The present invention relates generally to interleukin-2 proteins. More specifically, the present invention relates to methods of enhancing the circulating half-life of interleukin-2 proteins.

### BACKGROUND OF THE INVENTION

Interleukin-2 (IL-2) is an important cytokine which plays a role in the body's defense mechanism. For example, IL-2 is involved in the generation of antitumor immunity. In response to tumor antigens, helper T-cells secrete amounts of IL-2. The secreted IL-2 acts locally at the site of tumor antigen stimulation to activate cytotoxic T-cells (CTL) and natural killer cells (NK), thereby mediating systemic tumor cell destruction.

The use of interleukin-2 (IL-2) fusion proteins to treat human disease is well established. However, one limitation associated with using IL-2 fusion proteins is that they have a relatively short serum half-life. In fact, the initial half-life of IL-2 in vivo is about 6 to 12 minutes (Anderson et al., Clin. Pharmacokinet. 27(1):19-31 (1994), the teachings of which are hereby incorporated by reference).

Fusion proteins can be generated either by chemical or genetic manipulation using methods known in the art. With chemical conjugation, the joining process may occur at different sites on the molecules, and generally results in molecules with varying degrees of modification that can affect the function of one or both proteins. The use of genetic fusions, on the other hand, makes the joining process more consistent, resulting in the production of consistent end products that retain the function of both component proteins. See, for example, Gillies et al., Proc. Natl. Acad. Sci. USA 89: 1428-1432 (1992), the teachings of which are hereby incorporated by reference.

### SUMMARY OF THE INVENTION

The invention is based on the surprising observation that when two or more amino acids are altered in the N-terminal region of an IL-2 protein (e.g. human IL-2), the IL-2 protein has an extended serum half-life. Preferably, the amino acid changes involve replacing lysines within the first 10 amino acids of the N-terminal region of the IL-2 protein with non-lysine amino acids such as amino acids with uncharged side chains.

In one embodiment, lysine at position 8 (Lys₈) and lysine at position 9 (Lys₉) of the IL-2 protein are replaced with a non-lysine amino acid such as a hydrophobic amino acid. For example, Lys₈ and Lys₉ can be replaced with any one of the hydrophobic amino acids selected from the group consisting of tryptophan, phenylalanine, tyrosine, methionine, glycine, alanine, leucine, isoleucine and valine. In one embodiment, Lys₈ and Lys₉ can be replaced with the same hydrophobic amino acid, e.g., both Lys₈ and Lys₉ can be replaced with an alanine. Alternatively, Lys₈ and Lys₉ can be replaced with non-identical amino acids, e.g., Lys₈ can be replaced with a glycine and Lys₉ can be replaced with an alanine.

In another embodiment, the threonine at position 3 (Thr₃) of IL-2 is O-glycosylated. In some circumstances, O-glycosylation at Thr₃ serves to enhance the serum half-life of the IL-2 protein. Accordingly, in one embodiment, the Thr₃ of the IL-2 protein is not altered. In another embodiment, the Thr₃ of the IL-2 protein is altered to another amino acid which can be O-glycosylated such as a serine.

In one embodiment, the IL-2 protein is part of a fusion protein and includes a carrier protein. In one embodiment, the carrier protein is fused to the N-terminal portion of the IL-2 protein. A linker peptide may be inserted between the carrier protein and the IL-2 protein.

The carrier protein can be any polypeptide fused to the IL-2 protein. In one embodiment, the carrier protein is albumin, for example, human serum albumin. In another embodiment the carrier protein is an immunoglobulin (Ig) moiety, for example, the Ig moiety can include part of an Ig heavy chain.

In one embodiment, one or more amino acids at the C-terminal portion of the Ig moiety is replaced with a hydrophobic amino acid. For example, the Ig moiety is derived from an IgG sequence in which the C-terminal lysine residue is replaced. Preferably, the C-terminal lysine of an IgG sequence is replaced with a non-lysine amino acid, such as alanine, to further increase the serum half-life of the fusion protein. In another embodiment, the Ig moiety includes at least the CH2 domain of an IgG2 or an IgG4 constant region. In another embodiment, the Ig moiety comprises at least a portion of an IgG1 constant region where one or more amino acids selected from the group consisting of Leu₂₃₄, Leu₂₃₅, Gly₂₃₆, Gly₂₃₇, Asn₂₉₇, and Pro₃₃₁ are mutated or deleted. Preferably, one or more of these amino acids are replaced with a hydrophobic amino acid. In another embodiment, the Ig moiety comprises at least a portion of an IgG3 constant region where one or more amino acids selected from the group consisting of Leu₂₈₁, Leu₂₈₂, Gly₂₈₃, Gly₂₈₄, Asn₃₄₄, and Pro₃₇₈ are mutated or deleted. Preferably, one or more of these amino acids are replaced with a hydrophobic amino acid.

In another aspect, the invention relates to nucleic acid encoding an IL-2 protein of the invention; an expression vector containing the nucleic acid; or cell lines, e.g., myelomas, transfected with these constructs.

In another aspect, the invention relates to a method for preparing an IL-2 protein of the invention. The method includes inducing expression of the IL-2 protein described above, preferably in a suitable cell transfected with an expression vector containing the nucleic acid encoding the IL-2 protein of the invention, and obtaining the recombinant protein.

In another aspect, the invention relates to a composition including the IL-2 protein described above and a pharmaceutically acceptable carrier. The invention also relates to a method of treating a disease in a mammal by administering a pharmaceutical composition including the IL-2 protein of the present invention. In preferred embodiments, a composition of the invention is useful to treat a human with a disease relating to cancer, viral infections, or immune disorders. A composition of the present invention can also be used to enhance the growth (and proliferation) of specific cell types. In another embodiment, the present invention relates to a method of treating a patient by administering to the patient the nucleic acid encoding an IL-2 protein of the invention or a cell containing the nucleic acid.

The invention further features a method of screening a polypeptide, for example, a fusion protein such as an immunocytokine or an IL-2 fusion protein, for the extent of O-glycosylation present on the polypeptide. The method includes providing a polypeptide which has an O-glycosylation site and measuring the level of O-glycosylation. By comparing the level of O-glycosylation with a control the pharmacokinetic properties of the polypeptide can be determined. The control is a corresponding polypeptide, e.g., an immunocytokine, which is O-glycosylated and has known pharmacokinetic properties.

These and other objects, along with advantages and features of the invention disclosed herein, will be made more apparent from the description, drawings, and claims that follow.

### BRIEF DESCRIPTION OF THE DRAWINGS

FIG. 1 shows the pharmacokinetic behavior of various mutant KS-IL-2 fusion proteins as described in the Examples.

FIG. 2 shows the pharmacokinetic effect of various mutant KS-IL-2 fusion proteins when injected into Balb/C mice.

FIG. 3 depicts the amino acid sequence of a human IL-2 sequence including its leader peptide sequence, which is underlined (SEQ ID NO:1).

FIG. 4 depicts the amino acid sequence of a Macaca mulatta (rhesus monkey) IL-2 sequence including its leader peptide sequence, which is underlined (SEQ ID NO:2).

FIG. 5 depicts the amino acid sequence of a Macaca fascicularis IL-2 sequence including its leader peptide sequence, which is underlined (SEQ ID NO:3).

FIG. 6 depicts the amino acid sequence of a Cercocebus torquatus atys (sooty mangabey) IL-2 sequence including its leader peptide sequence, which is underlined (SEQ ID NO:4).

FIG. 7 depicts the amino acid sequence of a human serum albumin-IL-2 fusion protein with alterations in the IL-2 protein shown in bold (SEQ ID NO:5).

FIG. 8 depicts the amino acid sequence of a human gamma 4 constant region of IgG (SEQ ID NO:6).

FIG. 9 depicts the amino acid sequence of a human gamma 1 constant region of IgG (SEQ ID NO:7).

FIG. 10 depicts the amino acid sequence of a human gamma 2 constant region of IgG (SEQ ID NO:8).

### DETAILED DESCRIPTION OF THE INVENTION

The present invention is based on the finding that when more than one lysine residue in the N-terminal region of the IL-2 protein is replaced with a non-lysine residue, the protein exhibits an extended half-life. Indeed, replacing Lys₈ and Lys₉ of an IL-2 protein with non-lysine residues dramatically increases the serum half-life when compared to an IL-2 protein with no mutation or an IL-2 protein where only one lysine is replaced with a non-lysine residue. The present finding provides particularly therapeutically useful forms of IL-2.

In a preferred embodiment, the IL-2 protein is part of a fusion protein with a carrier protein. In one embodiment, the carrier protein is disposed towards the N-terminus of the fusion protein and the IL-2 protein is disposed towards the C-terminus. In this embodiment, the N-terminal region of the IL-2 protein, which contains the alterations in the lysine residues, occurs near the junction between the carrier protein and the IL-2 protein. In one embodiment, two or more lysines are altered in the first 10, 20, 30, 40, or 50 amino acids of the N-terminal region of the IL-2 protein.

As used herein, an “alteration” or “altered amino acid” refers to the replacement of an amino acid with another amino acid. In preferred embodiments, the alteration increases the hydrophobicity of the fusion protein's junction region. For example, the mutation replaces a charged or ionizable amino acid with a non-charged or hydrophobic amino acid (e.g., a Lys, Arg or other ionizable residue is replaced with an Ala, Leu, Gly, Tyr, Phe, Met, Trp or other non-charged or hydrophobic residue).

While not wishing to be bound by theory, it is believed that altering the lysines present in the N-terminal region of the IL-2 protein reduces the rate at which the IL-2 protein is proteolytically cleaved. It is believed that protease digestion may contribute to the disappearance of intact proteins from the body, including fusion proteins. By altering the lysines in the N-terminal region of the IL-2 protein this results in a change in the general conformation of the protein which is believed to limit access of the protease to their cleavage sites in the protein.

In another embodiment, the carrier protein is disposed towards the C-terminus of the fusion protein and the IL-2 protein is disposed towards the N-terminus.

The IL-2 protein can be directly linked to the carrier protein. Alternatively, the IL-2 protein can be linked to the carrier protein through a linker or spacer.

Interleukin-2

The invention includes an IL-2 protein which contains at least two amino acid substitutions in the N-terminal region of the protein, e.g., within the first 10, 20, 30, 40, 50, or 100 amino acids of the N-terminal region. Preferably, the two N-terminal lysines, Lys₈ and Lys₉, are substituted with non-lysine amino acids such as hydrophobic amino acids. Exemplary hydrophobic amino acids are selected from the group consisting of tryptophan, glycine, alanine, leucine, isoleucine and valine. In one embodiment, Lys₈ and Lys₉ can be replaced with identical hydrophobic amino acids, e.g., Lys₈ and Lys₉ can be replaced with alanines. Alternatively, Lys₈ and Lys₉ can be replaced with non-identical amino acids, e.g., Lys₈ can be replaced with a glycine and Lys₉ can be replaced with an alanine.

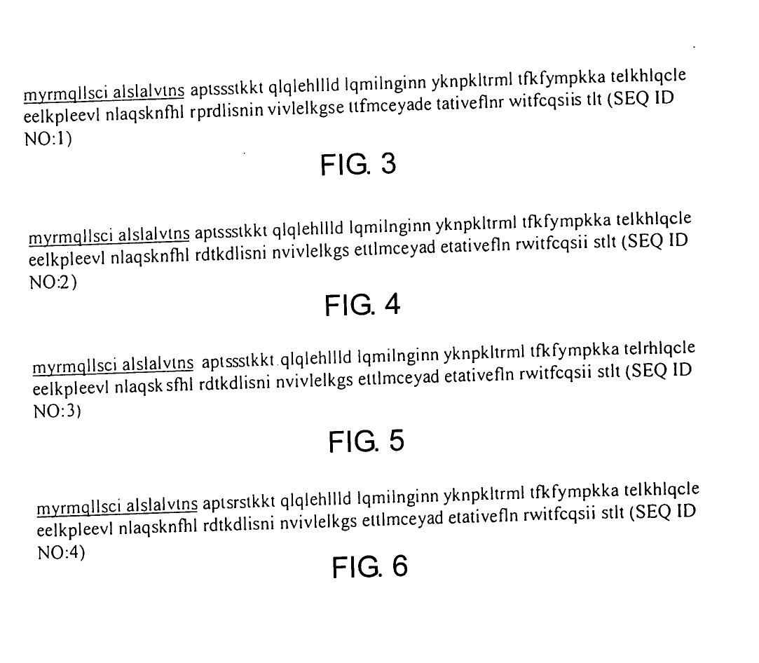

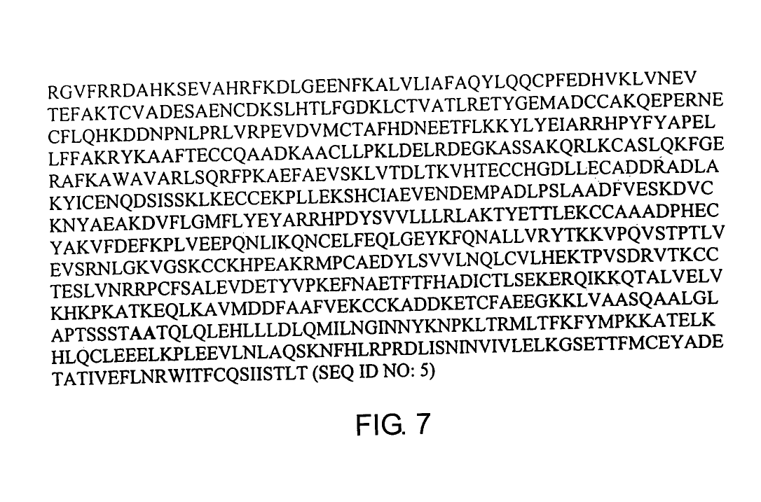

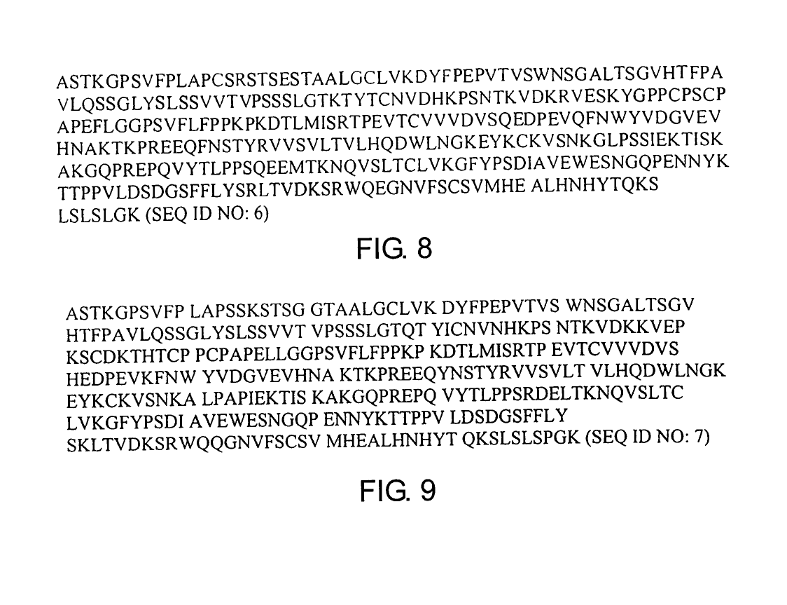

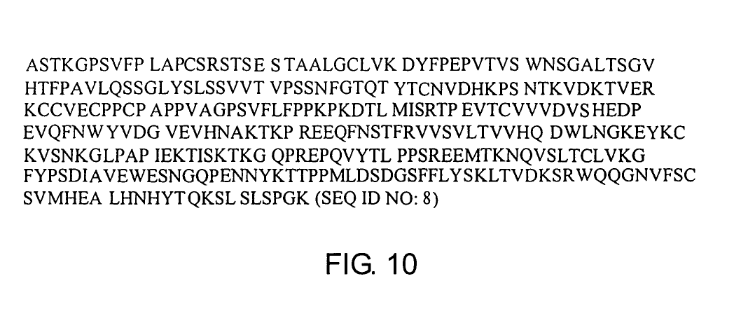

The terms “interleukin-2 protein” and “IL-2 protein” refer to an amino acid sequence of a recombinant or non-recombinant polypeptide having an amino acid sequence of: i) a wild-type or naturally-occurring allelic variant of an IL-2 polypeptide which has lysines at position 8 and position 9, ii) a biologically active fragment of an IL-2 polypeptide which has lysines at position 8 and position 9, iii) a biologically active analog of an IL-2 polypeptide which has lysines at position 8 and position 9, or iv) a biologically active variant of an IL-2 polypeptide which has lysines at position 8 and position 9. IL-2 polypeptides of the invention can be obtained from any species, e.g., primates such as human or monkey. IL-2 nucleic acid and amino acid sequences are well known in the art. For example, the human IL-2 sequence (Genbank accession number P01585; SEQ ID NO: 1) is shown in FIG. 3; the Macaca mulatta (rhesus monkey) IL-2 sequence (Genbank accession number P51498; SEQ ID NO:2) is shown in FIG. 4; the Macaca fascicularis IL-2 sequence (Genbank accession number Q29615; SEQ ID NO:3) is shown in FIG. 5; and the Cercocebus torquatus atys (sooty mangabey) IL-2 sequence (Genbank accession number P46649; SEQ ID NO:4) is shown in FIG. 6.

A “variant” of a human IL-2 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological or immunological activity can be found using computer programs well known in the art, for example, DNAStar software. The IL-2 proteins contemplated by the invention include IL-2 proteins, fragments of IL-2 proteins, variants or analogs thereof that retain IL-2 activity. A biologically-active or functionally-active IL-2 protein typically shares substantial amino acid sequence similarity or identity (e.g., at least about 55%, about 65%, about 75% identity, typically at least about 80% and most typically about 90-95% identity) with the corresponding sequences of wild-type, or naturally-occurring IL-2 protein and possesses one or more of the functions of wild-type IL-2 protein thereof. The activity of the IL-2 protein can be measured in a T-cell proliferation assay as described by Gillis et al. ((1978) J. Immunol. 120: 2027-2032, the teachings of which are hereby incorporated by reference) or using a cell-based assay as described in the examples section.

Carrier Protein

The carrier protein can be any polypeptide fused to an IL-2 protein. Examples of carrier proteins include those proteins with a long plasma half-life. Preferred carrier proteins are at least 50 amino acids, at least 100 amino acids, or at least 200 amino acids in length. Typically, proteins that exhibit an extended serum half-life are those proteins which have a high molecular weight, e.g., greater than 50,000 Daltons. Preferably, the carrier protein limits the proteolytic cleavage of the fusion protein. The circulating half-life of the IL-2 fusion protein can be measured by assaying the serum level of the fusion protein as a function of time.

In one embodiment, the carrier protein can also contain an alteration in its sequence, for example, preferably in the C-terminal portion of the carrier protein, e.g., within about 100 residues, more preferably within about 50 residues, or about 25 residues, and even more preferably within about 10 residues from the C-terminus of the carrier protein.

In one embodiment, the carrier protein is albumin, for example, human serum albumin (HSA). The genes coding for HSA are highly polymorphic and more than 30 different genetic alleles have been reported (Weitkamp L. R. et al., Ann. Hum. Genet. 37 (1973) 219-226, the teachings of which are hereby incorporated by reference). Alternatively, the albumin can be from any animal such as dog, chicken, duck, mouse or rat.

In another embodiment the carrier protein is an antibody. In general, an antibody-based IL-2 fusion protein of the invention comprises a portion of an immunoglobulin (Ig) protein joined to an IL-2 protein. Examples of immunoglobulins include IgG, IgM, IgA, IgD, and IgE.

The immunoglobulin protein or a portion of an immunoglobulin protein can include a variable or a constant domain. An immunoglobulin (Ig) chain preferably includes a portion of an immunoglobulin heavy chain, for example, an immunoglobulin variable region capable of binding a preselected cell-type. In a preferred embodiment, the Ig chain comprises a variable region specific for a target antigen as well as a constant region. The constant region may be the constant region normally associated with the variable region, or a different one, e.g., variable and constant regions from different species. In a more preferred embodiment, an Ig chain includes a heavy chain. The heavy chain may include any combination of one or more CH1, CH2, or CH3 domains. Preferably, the heavy chain includes CH1, CH2, and CH3 domains, and more preferably only CH2 and CH3 domains. In one embodiment, the portion of the immunoglobulin includes an Fv region with fused heavy and light chain variable regions.

In one embodiment, the carrier protein comprises an Fc portion of an immunoglobulin protein. As used herein, “Fc portion” encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

In the present invention, the Fc portion typically includes at least a CH2 domain. For example, the Fc portion can include, from N-terminus to C-terminus, hinge, CH2, and CH3 domains. Alternatively, the Fc portion can include all or a portion of the hinge region, the CH2 domain and/or the CH3 domain.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four y subclasses: γ1, γ2, γ3, and γ4, also known as IgG1, IgG2, IgG3, and IgG4, respectively.

IgG molecules interact with multiple classes of cellular receptors including three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains. The serum half-life of an antibody is influenced by the ability of that antibody to bind to an Fc receptor (FcR). Similarly, the serum half-life of immunoglobulin fusion proteins is also influenced by the ability to bind to such receptors (Gillies S D et al., (1999) Cancer Res. 59:2159-66, the teachings of which are hereby incorporated by reference). Compared to those of IgG1, CH2 and CH3 domains of IgG2 and IgG4 have biochemically undetectable or reduced binding affinity to Fc receptors. It has been reported that immunoglobulin fusion proteins containing CH2 and CH3 domains of IgG2 or IgG4 had longer serum half-lives compared to the corresponding fusion proteins containing CH2 and CH3 domains of IgG1 (U.S. Pat. No. 5,541,087; Lo et al., (1998) Protein Engineering, 11:495-500, the teachings of which are hereby incorporated by reference). Accordingly, preferred CH2 and CH3 domains for the present invention are derived from an antibody isotype with reduced receptor binding affinity and effector functions, such as, for example, IgG2 or IgG4. More preferred CH2 and CH3 domains are derived from IgG2.

The hinge region is normally located C-terminal to the CH1 domain of the heavy chain constant region. In the IgG isotypes, disulfide bonds typically occur within this hinge region, permitting the final tetrameric molecule to form. This region is dominated by prolines, serines and threonines. When included in the present invention, the hinge region is typically at least homologous to the naturally-occurring immunoglobulin region that includes the cysteine residues to form disulfide bonds linking the two Fc moieties. Representative sequences of hinge regions for human and mouse immunoglobulins can be found in Borrebaeck, C. A. K., ed., (1992) ANTIBODY ENGINEERING, A PRACTICAL GUIDE, W. H. Freeman and Co., the teachings of which are hereby incorporated by reference. Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8, the teachings of which are hereby incorporated by reference).

In accordance with the present invention, the Fc portion can contain CH2 and/or CH3 domains and a hinge region that are derived from different antibody isotypes, i.e., a hybrid Fc portion. For example, in one embodiment, the Fc portion contains CH2 and/or CH3 domains derived from IgG2 or IgG4 and a mutant hinge region derived from IgG1. Alternatively, a mutant hinge region from another IgG subclass is used in a hybrid Fc portion. For example, a mutant form of the IgG4 hinge that allows efficient disulfide bonding between the two heavy chains can be used. A mutant hinge can also be derived from an IgG2 hinge in which the first two cysteines are each mutated to another amino acid. Such hybrid Fc portions facilitate high-level expression and improve the correct assembly of the Fc fusion proteins. Assembly of such hybrid Fc portions has been described in U.S. Patent Application Publication No. 20030044423, the disclosure of which is hereby incorporated by reference.

In some embodiments, the Fc portion contains amino acid modifications that generally extend the serum half-life of an Fc fusion protein. Such amino acid modifications include mutations substantially decreasing or eliminating Fc receptor binding or complement fixing activity. For example, the glycosylation site within the Fc portion of an immunoglobulin heavy chain can be removed. In IgG1, the glycosylation site is Asn297. In other immunoglobulin isotypes, the glycosylation site corresponds to Asn297 of IgG1. For example, in IgG2 and IgG4, the glycosylation site is the asparagine within the amino acid sequence Gln-Phe-Asn-Ser. Accordingly, a mutation of Asn297 of IgG1 removes the glycosylation site in an Fc portion derived from IgG1. In one embodiment, Asn297 is replaced with Gln. Similarly, in IgG2 or IgG4, a mutation of asparagine within the amino acid sequence Gln-Phe-Asn-Ser removes the glycosylation site in an Fc portion derived from IgG2 or IgG4 heavy chain. In one embodiment, the asparagine is replaced with a glutamine. In other embodiments, the phenylalanine within the amino acid sequence Gln-Phe-Asn-Ser is further mutated to eliminate a potential non-self T-cell epitope resulting from asparagine mutation. For example, the amino acid sequence Gln-Phe-Asn-Ser within an IgG2 or IgG4 heavy chain can be replaced with a Gln-Ala-Gln-Ser amino acid sequence.

It has also been observed that alteration of amino acids near the junction of the Fc portion and the non-Fc portion can dramatically increase the serum half-life of the Fc fusion protein (PCT publication WO 01/58957, the disclosure of which is hereby incorporated by reference). Accordingly, the junction region of an Fc-IL-2 fusion protein of the present invention can contain alterations that, relative to the naturally-occurring sequences of an immunoglobulin heavy chain and an IL-2 protein, preferably lie within about 10 amino acids of the junction point. These amino acid changes can cause an increase in hydrophobicity by, for example, changing the C-terminal lysine of the Fc portion to a hydrophobic amino acid such as alanine or leucine.

In other embodiments, the Fc portion contains amino acid alterations of the Leu-Ser-Leu-Ser segment near the C-terminus of the Fc portion of an immunoglobulin heavy chain. The amino acid substitutions of the Leu-Ser-Leu-Ser segment eliminate potential junctional T-cell epitopes. In one embodiment, the Leu-Ser-Leu-Ser amino acid sequence near the C-terminus of the Fc portion is replaced with an Ala-Thr-Ala-Thr amino acid sequence. In other embodiments, the amino acids within the Leu-Ser-Leu-Ser segment are replaced with other amino acids such as glycine or proline. Detailed methods of generating amino acid substitutions of the Leu-Ser-Leu-Ser segment near the C-terminus of an IgG1, IgG2, IgG3, IgG4, or other immunoglobulin class molecule have been described in U.S. Patent Application Publication No. 20030166877, the disclosure of which is hereby incorporated by reference.

According to the invention, an antibody-based fusion protein with an enhanced in vivo circulating half-life can be further enhanced by modifying within the Fc portion itself. These may be residues including or adjacent to Ile 253, His 310 or His 435 or other residues that can affect the ionic environments of these residues when the protein is folded in its 3-dimensional structure. The resulting proteins can be tested for optimal binding at pH 6 and at pH 7.4-8 and those with high levels of binding at pH 6 and low binding at pH 8 are selected for use in vivo. Such mutations can be usefully combined with the junction mutations of the invention.

In another embodiment of the invention, the binding affinity of fusion proteins for FcRp is optimized by alteration of the interaction surface of the Fc moiety that contacts FcRp. The important sequences for the binding of IgG to the FcRp receptor have been reported to be located in the CH2 and CH3 domains. According to the invention, alterations of the fusion junction in a fusion protein are combined with alterations of the interaction surface of Fc with FcRp to produce a synergistic effect. In some cases it may be useful to increase the interaction of the Fc moiety with FcRp at pH 6, and it may also be useful to decrease the interaction of the Fc moiety with FcRp at pH 8. Such modifications include alterations of residues necessary for contacting Fc receptors or altering others that affect the contacts between other heavy chain residues and the FcRp receptor through induced conformational changes. Thus, in a preferred embodiment, an antibody-based fusion protein with enhanced in vivo circulating half-life is obtained by first linking the coding sequences of an Ig constant region and a second, non-immunoglobulin protein and then introducing a mutation (such as a point mutation, a deletion, an insertion, or a genetic rearrangement) in an IgG constant region at or near one or more amino acid selected from Ile₂₅₃, His₃₁₀ and His₄₃₅. The resulting antibody-based fusion proteins have a longer in vivo circulating half-life than the unmodified fusion proteins.

In certain circumstances it is useful to mutate certain effector functions of the Fc moiety. For example, complement fixation may be eliminated. Alternatively or in addition, in another set of embodiments the Ig component of the fusion protein has at least a portion of the constant region of an IgG that has reduced binding affinity for at least one of FcγRI, FcγRII or FcγRIII. For example, the gamma4 chain of IgG may be used instead of gamma1. The alteration has the advantage that the gamma4 chain results in a longer serum half-life, functioning synergistically with one or more mutations at the fusion junction. Similarly, IgG2 may also be used instead of IgG1. In an alternative embodiment of the invention, a fusion protein includes a mutant IgG1 constant region, for example an IgG1 constant region having one or more mutations or deletions of Leu₂₃₄, Leu₂₃₅, Gly₂₃₆, Gly₂₃₇, Asn₂₉₇, or Pro₃₃₁. In a further embodiment of the invention, a fusion protein includes a mutant IgG3 constant region, for example an IgG3 constant region having one or more mutations or deletions of Leu₂₈₁, Leu₂₈₂, Gly283, Gly₂₈₄, Asn₃₄₄, or Pro₃₇₈. However, for some applications, it may be useful to retain the effector function that accompanies Fc receptor binding, such as ADCC.

In another preferred embodiment, the carrier protein of the fusion protein is a protein toxin. Preferably, the toxin-IL-2 fusion protein of the present invention displays the toxic activity of the protein toxin.

In some embodiments, the carrier protein of the fusion protein is a hormone, neurotrophin, body-weight regulator, serum protein, clotting factor, protease, extracellular matrix component, angiogenic factor, anti-angiogenic factor, or another secreted protein or secreted domain. For example, CD26, IgE receptor, polymeric IgA receptor, other antibody receptors, Factor VIII, Factor IX, Factor X, TrkA, PSA, PSMA, Flt-3 Ligand, endostatin, angiostatin, and domains of these proteins.

In other embodiments, the carrier protein is a non-human or non-mammalian protein. For example, HIV gp120, HIV Tat, surface proteins of other viruses such as adenovirus, and RSV, other HIV components, parasitic surface proteins such as malarial antigens, and bacterial surface proteins are preferred. These non-human proteins may be used, for example, as antigens, or because they have useful activities. For example, the carrier polypeptide may be streptokinase, staphylokinase, urokinase, tissue plasminogen activator, or other proteins with useful enzymatic activities.

In certain embodiments, the carrier protein is a cytokine. The term “cytokine” is used herein to describe naturally occurring or recombinant proteins, analogs thereof, and fragments thereof which elicit a specific biological response in a cell which has a receptor for that cytokine. Preferably, cytokines are proteins that may be produced and excreted by a cell. Preferred cytokines include interleukins such as IL-2, IL-4, IL-5, IL-6, IL-7, IL-10, IL-12, IL-13, IL-14, IL-15, IL-16 and IL-18, hematopoietic factors such as granulocyte-macrophage colony stimulating factor (GM-CSF), granulocyte colony stimulating factor (G-CSF) and erythropoeitin, tumor necrosis factors (TNF) such as TNFα, lymphokines such as lymphotoxin, regulators of metabolic processes such as leptin, interferons such as interferon α, interferon β, and interferon γ, and chemokines.

Spacer

In an optional embodiment, a spacer or linker peptide is inserted between the carrier protein and the IL-2 protein. The spacer or linker peptide is preferably non-charged and more preferably non-polar or hydrophobic. The length of a spacer or linker peptide is preferably between 1 and about 100 amino acids, more preferably between 1 and about 50 amino acids, or between 1 and about 25 amino acids, and even more preferably between 1 and about 15 amino acids. In another embodiment of the invention, the carrier protein and the IL-2 protein are joined via a spacer or linker peptide. In an alternative embodiment of the invention, the carrier protein and IL-2 protein are separated by a synthetic spacer, for example a PNA spacer, that is preferably non-charged, and more preferably non-polar or hydrophobic.

The linker can be designed to include no protease cleavage site. Furthermore, the linker can contain an N-linked or an O-linked glycosylation site to sterically inhibit proteolysis. Accordingly, in one embodiment, the linker contains an Asn-Ala-Thr amino acid sequence.

Additional suitable linkers are disclosed in Robinson et al., (1998), Proc. Natl. Acad. Sci. USA; 95, 5929; and U.S. application Ser. No. 09/708,506, the disclosures of both of which are hereby incorporated by reference.

O-Glycosylation and Methods of Screening the Pharmacokinetic Properties of Proteins

The extent of O-glycosylation of an amino acid was found to have an influence on the circulating half-life of the protein. For example, the threonine at position 3 (Thr₃) of IL-2 is O-glycosylated and by substituting Thr₃ of IL-2 the resulting Ig-IL-2-fusion protein has a reduced serum half-life. Not wishing to be bound by theory, it may be that the junction between the cytokine and its fusion partner is particularly susceptible to proteolytic cleavage. It is believed that the presence of a bulky glycan on an amino acid side chain near the junction site may reduce access of proteases to the junction. Accordingly, in one embodiment, in order to extend the half-life of the protein, it is preferable not to mutate amino acids at the junction which can be O-glycosylated, or preferable to introduce amino acids which can be glycosylated into the junction. In another embodiment, it may be preferable to introduce a threonine or a serine at the junction site.

The extent of O-glycosylation of the protein, e.g., the immunocytokine, depends on the cell line and the culturing conditions used to produce the protein. Since the extent of O-glycosylation affects the half-life of the protein, it is preferable when producing a protein to be able to measure the extent of O-glycosylation to predict the serum half-life of the protein batch. Moreover, since the protein is used in the treatment of diseases, it is preferable that different batches of produced protein have uniform properties. This can be achieved by comparing the extent of O-glycosylation of the produced protein (also referred to as the “test protein”) with a reference control. A reference control is a protein which is substantially the same as the test protein, has a predetermined amount of O-glycosylation and whose serum half-life is known. By comparing the test protein with the reference control, the serum half-life of the protein can be determined or estimated. Alternatively, as a means of ensuring that the test proteins have batch-to-batch uniformity, batches of test proteins that do not have an equivalent extent of O-glycosylation as the reference control can be discarded.

The invention further provides methods of screening the pharmacokinetic properties of proteins, e.g., immunocytokines, e.g., an Ig-IL-2 fusion protein, by measuring the extent of O-glycosylation. In one embodiment, the method includes producing an immunocytokine of interest in a cell line, e.g., a mammalian cell line, such as, for example, CHO, BHK, NIH 293, or PERC6. The immunocytokine is then isolated from the cell line and the extent of O-glycosylation measured, e.g., using methods such as periodate oxidation/Schiff's staining of SDS-PAGE gels to identify a protein as O-glycosylated or using Western blotting by immunostaining methods which have been developed and commercialized by several suppliers (e.g., Oxford GlycoSystems, Boehringer-Mannheim). Alternatively, in one example, the extent of O-glycosylation in a sample of a protein, such as a fusion protein, can be measured as follows. Wells in a microtiter plate are coated with the immunocytokine to be analyzed. A peanut lectin (PNA, peanut agglutinin, Roche Diagnostics GMBH, Mannheim Germany) that has been labeled, for example by biotinylation, is added to the analyte-coated well, and allowed to adsorb to the sample. Excess unbound lectin is removed by washing. A secondary detection molecule, such as streptavidin conjugated to horseradish peroxidase, is added. The bound complexes are washed and the amount of bound secondary detection molecule is determined by standard procedures. In some cases it is useful to normalize the level of detected O-glycan to the level of bound analyte as detected by a protein-directed antibody. This lectin-based assay is appropriate as a release assay for batch-testing of material for human use. Alternatively, a Western blot-type assay is used in which the labeled lectin is used as a probe.

In another embodiment, in order to eliminate having to characterize the glycosylation status of an immunocytokine, it may be advantageous to remove amino acids which can be O-glycosylated. For example, in one embodiment, the Thr₃ of IL-2 can be substituted with an amino acid which can not be O-glycosylated such as an alanine. In this embodiment, immunocytokines lacking an amino acid susceptible to O-glycosylation show better batch-to-batch uniformity.

Administration

Pharmaceutical Compositions and Administration Routes

The IL-2 proteins of the invention can be used to treat viral infections, immune disorders, and to enhance the growth (including proliferation) of specific cell types. Moreover, the IL-2 proteins can be used as an anticancer agent for the treatment of cancers including, but not limited to, bladder cancer, lung cancer, brain cancer, breast cancer, skin cancer, and prostate cancer. Thus, the present invention also provides pharmaceutical compositions containing the IL-2 protein produced according to the present invention.

The therapeutic compositions containing IL-2 fusion proteins produced according to the present invention can be administered to a mammalian host by any route. Thus, as appropriate, administration can be oral or parenteral, including intravenous and intraperitoneal routes of administration. Medicaments can be prepared in the form of tablets, capsules, pills, granules, sublingual tablet, dragees, ointment, suppository, syrup, and suspension. In addition, administration can be by periodic injections of a bolus of the therapeutics or can be made more continuous by intravenous or intraperitoneal administration from a reservoir which is external (e.g., an i.v. bag). In certain embodiments, the therapeutics of the instant invention can be pharmaceutical-grade. That is, certain embodiments comply with standards of purity and quality control required for administration to humans. Veterinary applications are also within the intended meaning as used herein.

The formulations, both for veterinary and for human medical use, of the therapeutics according to the present invention typically include such therapeutics in association with a pharmaceutically-acceptable carrier and optionally other ingredient(s). The carrier(s) can be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, diluents, disintegrators, bases, isotonic agents, binders, buffers, adsorbents, lubricants, solvents, stabilizing agents, antioxidants, preservatives, sweetening agents, emulsifying agents, coloring agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds also can be incorporated into the compositions. The formulations can conveniently be presented in dosage unit form and can be prepared by any of the methods well known in the art of pharmacy/microbiology. In general, some formulations are prepared by bringing the therapeutics into association with a liquid carrier or a finely divided solid carrier or both, and then, if necessary, shaping the product into the desired formulation.

A pharmaceutical composition of the invention is formulated to be compatible with its intended route of administration. Examples of routes of administration include oral or parenteral, e.g., intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., 1990. Formulations for parenteral administration also can include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these therapeutics include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the therapeutics which can be in microcrystalline form, for example, in the form of an aqueous microcrystalline suspension. Liposomal formulations or biodegradable polymer systems can also be used to present the therapeutics for both intra-articular and ophthalmic administration.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and filsidic acid derivatives. Transmucosal administration can be accomplished through the use of nasal sprays or suppositories. For transdermal administration, the therapeutics typically are formulated into ointments, salves, gels, or creams as generally known in the art.

In one embodiment, the therapeutics are prepared with carriers that will protect against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,81 1, the disclosure of which is hereby incorporated by reference. Microsomes and microparticles also can be used.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Determining Therapeutically-Effective Amount of an IL-2 Protein and Dosing Frequency

Generally, the therapeutics containing IL-2 proteins produced according to the present invention can be formulated for parenteral or oral administration to humans or other mammals, for example, in therapeutically effective amounts, e.g., amounts which provide appropriate concentrations of the drug to target tissue for a time sufficient to induce the desired effect, e.g., the desired immune response. The amount will vary from one individual to another and will depend upon a number of factors, including the overall physical condition of the patient, severity and the underlying cause of disease.

A therapeutically effective amount of an IL-2 protein may be readily ascertained by one skilled in the art. The effective concentration of the IL-2 protein of the invention that is to be delivered in a therapeutic composition will vary depending upon a number of factors, including the final desired dosage of the drug to be administered and the route of administration. The preferred dosage to be administered also is likely to depend on such variables as the type and extent of disease or indication to be treated, the overall health status of the particular patient, the relative biological efficacy of the therapeutics delivered, the formulation of the therapeutics, the presence and types of excipients in the formulation, and the route of administration. In some embodiments, the therapeutics of this invention can be provided to an individual using typical dose units deduced from the mammalian studies using non-human primates and rodents. As described above, a dosage unit refers to a unitary dose which is capable of being administered to a patient, and which can be readily handled and packed, remaining as a physically and biologically stable unit dose comprising either the therapeutics as such or a mixture of it with solid or liquid pharmaceutical diluents or carriers.

Medicaments that contain the IL-2 proteins of the invention can have a concentration of 0.01 to 100% (w/w), though the amount varies according to the dosage form of the medicaments.

Administration dose depends on the body weight of the patients, the seriousness of the disease, and the doctor's opinion. However, it is generally advisable to administer about 0.01 to about 10 mg/kg body weight a day, preferably about 0.02 to about 2 mg/kg in case of injection.

Daily dose can be administered once or several times according to seriousness of the disease and doctor's opinion.

Compositions of the invention are useful when coadministered with angiogenesis inhibitors such as those disclosed in PCT/US99/08335 (WO 99/52562) or prostaglandin inhibitors such as those disclosed in PCT/US99/08376 (WO 99/53958), the teachings of which are hereby incorporated by reference. Methods and compositions of the invention can also be used in multiple cytokine protein complexes such as those disclosed in PCT/US00/21715, the teachings of which are hereby incorporated by reference. Methods and compositions of the invention are also useful in combination with other mutations disclosed in PCT/US99/03966 (WO 99/43713) that increase the circulating half-life of a fusion protein, the teachings of which are hereby incorporated by reference.

It is understood that the dosing frequencies actually used may vary somewhat from the frequencies disclosed herein due to variations in responses by different individuals to IL-2 and its analogs; the term “about” is intended to reflect such variations.

Additionally, the therapeutics of the present invention can be administered alone or in combination with other molecules known to have a beneficial effect on the particular disease or indication of interest. By way of example only, useful cofactors include symptom-alleviating cofactors, including antiseptics, antibiotics, antiviral and antifungal agents and analgesics and anesthetics.

Prodrug

Therapeutics of the invention also include “prodrug” derivatives. The term prodrug refers to a pharmacologically inactive (or partially inactive) derivative of a parent molecule that requires biotransformation, either spontaneous or enzymatic, within the organism to release or activate the active component. Prodrugs are variations or derivatives of the therapeutics of the invention which have groups cleavable under metabolic conditions. Prodrugs become the therapeutics of the invention which are pharmaceutically active in vivo, when they undergo solvolysis under physiological conditions or undergo enzymatic degradation. Prodrug of this invention can be called single, double, triple, and so on, depending on the number of biotransformation steps required to release or activate the active drug component within the organism, and indicating the number of functionalities present in a precursor-type form. Prodrug forms often offer advantages of solubility, tissue compatibility, or delayed release in the mammalian organism (see, Bundgard, (1985) Design of Prodrugs, pp. 7-9, 21-24, Elsevier, Amsterdam; Silverman, (1992) The Organic Chemistry of Drug Design and Drug Action, pp. 352-401, Academic Press, San Diego, Calif., the teachings of both of which are hereby incorporated by reference). Moreover, the prodrug derivatives according to this invention can be combined with other features to enhance bioavailability.

In Vivo Expression

The IL-2 protein of the present invention can be provided by in vivo expression methods. For example, a nucleic acid encoding an IL-2 protein can be advantageously provided directly to a patient suffering from cancer, viral infections, immune disorders, or other diseases, or may be provided to a cell ex vivo, followed by administration of the living cell to the patient. In vivo gene therapy methods known in the art include providing purified DNA (e.g., as in a plasmid), providing the DNA in a viral vector, or providing the DNA in a liposome or other vesicle (see, for example, U.S. Pat. No. 5,827,703, disclosing lipid carriers for use in gene therapy, and U.S. Pat. No. 6,281,010, providing adenoviral vectors useful in gene therapy, the teachings of both of which are hereby incorporated by reference).

Methods for treating disease by implanting a cell that has been modified to express a recombinant protein are also well known. See, for example, U.S. Pat. No. 5,399,346, disclosing methods for introducing a nucleic acid into a primary human cell for introduction into a human, the teachings of which are hereby incorporated by reference.

In vivo expression methods are particularly useful for delivering a protein directly to targeted tissues or cellular compartment without purification. In the present invention, gene therapy using the sequence encoding IL-2 fusion protein can find use in a variety of disease states, disorders and states of cancer, viral infections, immune disorders, and other cell proliferation associated diseases. A nucleic acid sequence coding for an IL-2 fusion protein can be inserted into an appropriate transcription or expression cassette and introduced into a host mammal as naked DNA or complexed with an appropriate carrier. Monitoring of the production of active IL-2 fusion protein can be performed by nucleic acid hybridization, ELISA, western hybridization, and other suitable methods known to ordinary artisan in the art.

It has been found that a plurality of tissues can be transformed following systemic administration of transgenes. Expression of exogenous DNA following intravenous injection of a cationic lipid carrier/exogenous DNA complex into a mammalian host has been shown in multiple tissues, including T lymphocytes, reticuloendothelial system, cardiac endothelial cells lung cells, and bone marrow cells, e.g., bone marrow-derived hematopoietic cells.

The in vivo gene therapy delivery technology as described in U.S. Pat. No. 6,627,615, the entire disclosure of which is hereby incorporated by reference, is non-toxic in animals and transgene expression has been shown to last for at least 60 days after a single administration. The transgene does not appear to integrate into host cell DNA at detectable levels in vivo as measured by Southern analysis, suggesting that this technique for gene therapy will not cause problems for the host mammal by altering the expression of normal cellular genes activating cancer-causing oncogenes, or turning off cancer-preventing tumor suppressor genes.

Non-limiting methods for synthesizing useful embodiments of the invention are described in the Examples herein, as well as assays useful for testing pharmacokinetic activities in pre-clinical in vivo animal models. The preferred gene construct encoding a chimeric chain includes, in 5′ to 3′ orientation, a DNA segment which encodes at least a portion of a carrier protein and DNA which encodes an IL-2 protein where the lysines at position 8 and 9 are replaced with non-lysine residues. The fused gene is assembled in or inserted into an expression vector for transfection of the appropriate recipient cells where it is expressed.

The invention is illustrated further by the following non-limiting examples.

### EXAMPLES

### Example 1

### Pharmacokinetic Profiles of Antibody-IL-2 Fusion Proteins

This example describes the effect of altering the lysines at the N-terminal region of IL-2 on the serum half-life of the antibody-IL-2 fusion protein.

Expression plasmids encoding the following antibody-IL-2 fusion proteins were constructed by standard molecular biology techniques:
Antibody(Ala [-1])-IL-2(Thr3 Ala8 Ala9)
Antibody(Ala [-1])-IL-2(Thr3 Lys8 Lys9)
Antibody(Ala [-1])-IL-2(Ala3 Lys8 Lys9)
Antibody(Lys [-1])-IL-2(Ala3 Lys8 Lys9)

In these particular cases, the antibody V regions were derived from the anti-EpCAM antibody KS-1/4 and various mutations were introduced to lessen the immunogenicity of the V regions in humans.

The construction of an expression vector encoding the Antibody(Ala [-1])-IL-2(Thr3 Lys8 Lys9) protein was performed as follows, and illustrates the general strategies used to construct the other variants described above. Construction strategies for these other variants will be readily apparent to those skilled in the art of plasmid construction.

For example, the plasmid pdHL7-KS-IL-2 was digested with XmaI and PvuII. This plasmid is an expression vector for KS-1/4-based immunocytokines and contains a unique SmaI/XmaI site near the end of the antibody heavy chain constant region coding sequence, as well as a unique PvuII site that occurs naturally in the human IL-2 coding sequence. Related pdHL7-based plasmids are discussed in U.S. Patent Application Publication No. 20030157054, the disclosure of which is hereby incorporated by reference. The following synthetic oligonucleotides were hybridized and then ligated to the XmaI, PvuII-digested vector.

SEQ ID NO: 9

CCGGGTGCCGCCCCAACTTCAAGTAGTACTGCCGCCACAG

SEQ ID NO: 10

CTGTGTGGCGGCAGTACTACTTGAAGTTGGGGCGGCAC

The full DNA sequence encoding the light chain and the heavy chain-IL-2 fusion protein are given below as SEQ ID NO:11 and SEQ ID NO:12.

SEQ ID NO: 11 DNA sequence encoding mature light chain of KS-IL-2 (K8A K9A). Lower case letters indicate introns.

GAGATCGTGCTGACCCAGTCCCCCGCCACCCTGTCCCTGTCCCCCGGCGAGCGCGTG
ACCCTGACCTGCTCCGCCTCCTCCTCCGTGTCCTACATGCTGTGGTACCAGCAGAAG
CCAGGATCCTCGCCCAAACCCTGGATTTTTGACACATCCAACCTGGCTTCTGGATTC
CCTGCTCGCTTCAGTGGCAGTGGGTCTGGGACCTCTTACTCTCTCATAATCAGCAGC
ATGGAGGCTGAAGATGCTGCCACTTATTACTGCCATCAGCGGAGTGGTTACCCGTAC
ACGTTCGGAGGGGGGACCAAGCTGGAAATAAAACgtaagatcccgcaattctaaactctgagggggtcg
gatgacgtggccattctttgcctaaagcattgagtttactgcaaggtcagaaaagcatgcaaagccctcagaatggctgcaaagagctccaa
caaaacaatttagaactttattaaggaatagggggaagctaggaagaaactcaaaacatcaagattttaaatacgcttcttggtctccttgctat
aattatctgggataagcatgctgttttctgtctgtccctaacatgccctgtgattatccgcaaacaacacacccaagggcagaactttgttactta
aacaccatcctgtttgcttctttcctcagGAACTGTGGCTGCACCATCTGTCTTCATCTTCCCGCCATCT
GATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTC
CCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGTGTT
AG

SEQ ID NO:12 DNA sequence encoding mature heavy chain-IL2 fusion moiety of KS-IL2 (K8A K9A). Lower case letters indicate introns and a 3′ non-coding region sequence that includes a convenient XhoI site. Underlined are XmaI and PvuII sites, between which mutations of the invention have been introduced.

CAGATCCAGTTGGTGGAGTCTGGAGCTGAGGTGAAGAAGCCTGGAGAGACAGTCAAGATCTCCTGCA
AGGCTTCTGGGTATACCTTCACAAACTATGGAATGAACTGGGTGAAGCAGACTCCAGGAAAGGGTTTA
AAGTGGATGGGCTGGATAAACACCTACACTGGAGAACCAACATATGCTGATGACTTCAAGGGACGGT
TTGCCTTCTCTTTGGAAACCTCTACCAGCACTGCCTTTTTGCAGATCAACAATCTCAGAAGTGAGGACA
CGGCTACATATTTGTGTGTAAGATTTATTTCTAAGGGGGACTACTGGGGTGAAGGAACGTCAGTCACC
GTCTCCTCAGgtaagctttctggggcaggccaggcctgaccttggctttggggcagggagggggctaaggtgaggcaggtggcgccagccaggtgcacacc
caatgcccatgagcccagacactggacgctgaacctcgcggacagttaagaacccaggggcctctgcgccctgggcccagctctgtcccacaccgcggtcacatggc
accacctctcttgcagCCTCCACGAAGGGCCCATCGGTCTTCCCCCTGGCACCCTCCTCCAAGAGCACGTGTGGG
GGCACAGCGGCCCTGGGCTGCCTGGTCAAGGACTACTTCCCCGAACCGGTGACGGTGTGGTGGAACTC
AGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACCCAGACCTACATCTGCAACGTGAATCACAAGC
CCAGCAACACGAAGGTGGACAAGAGAGTTGgtgagaggccagcacagggagggagggtgtctgctggaagccaggctcagcgctcctg
cctggacgcatcccggctatgcagtcccagtccagggcagcaaggcaggccccgtctgcctcttcacccggaggcctctgcccgccccactcatgctcagggagagg
gtcttctggctttttccccaggctctgggcaggcacaggctaggtgcccctaacccaggccctgcacacaaaggggcaggtgctgggctcagacctgccaagagccat
atccgggaggaccctgcccctgacctaagcccaccccaaaggccaaactctccactccctcagctcggacaccttctctcctcccagattccagtaactcccaatcttctct
ctgcagAGCCCAAATCTTGTGACAAAACTGACACATGCCCACCGTGCGCAGgtaagccagcccaggcctcgccctccagct
caaggcgggacaggtgccctagagtagcctgcatccagggacaggccccagccgggtgctgacacgtccacctccatctcttcctcagCACCTGAACTCC
TGGGGGGACCGTCAGTCTTCCTCTTCCCCGCAAAACCCAAGGACACCCTCATGATCTCCCGGACCCCTG
AGGTCACATGCGTGGTGGTGGACGTGAGCGACGAAGACCCTGAGGTCAAGTTCAACTGGTACGTGGA
CGGCGTGGAGGTGCATAATGCCAAGACAAAGCGGCGGGAGGAGCAGTACAACAGCACGTACCGTGTG
GTCAGCGTGCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGgtgggacccgtggggtgcgagggccacatgg
acagaggccggctcggcccaccctctgccctgagagtgaccgctgtaccaacctctgtccctacagGGCAGCCCCGAGAACCACAGGTG
TACACCCTGCCCCCATCACGGGAGGAGATGACCAAGAACCAGGTCAGCCTGACCTG
CCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCC
CTGTCCCCGGGTAAAGCCCCAACTTCAAGTAGTACTGCCGCCACACAGCTGCAACTG
GAGCATCTCCTGCTGGATCTCCAGATGATTCTGAATGGAATTAACAACTACAAGAAT
CCCAAACTCACCAGGATGCTCACATTCAAGTTCTACATGCCCAAGAAGGCCACAGA
GCTCAAACATCTCCAGTGTCTAGAGGAGGAACTCAAACCTCTGGAGGAAGTGCTAA
ACCTCGCTCAGAGCAAAAACTTCCACTTAAGACCTAGGGACTTAATCAGCAATATCA
ACGTAATAGTTCTGGAACTAAAGGGATCCGAAACAACATTCATGTGTGAATATGCTG
ATGAGACAGCAACCATTGTAGAATTCCTAAACAGATGGATTACCTTTTGTCAAAGCA
TCATCTCAACACTAACTTGAtaattaagtgctcgag

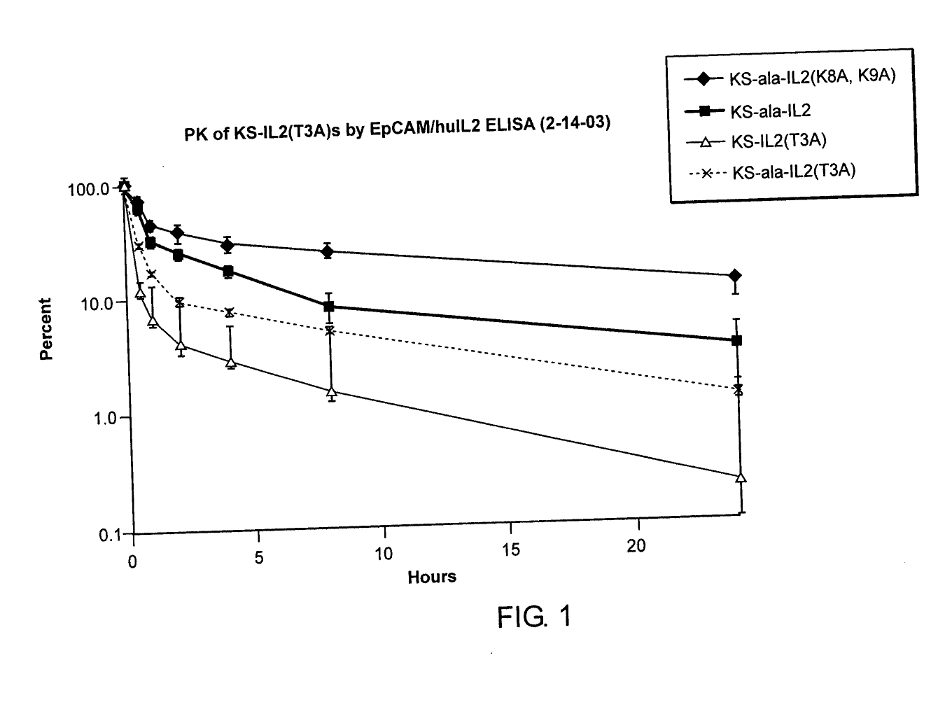

The proteins were purified from tissue-culture supernatant and their pharmacokinetic properties were studied. Results are shown in FIG. 1. The results show that the serum half-life of the antibody-IL-2 fusion protein containing mutations at both lysines 8 and 9 of IL-2 demonstrate profound improvements in serum half life. Moreover, it was observed that threonine 3 of IL-2 also affected the half-life of the protein.

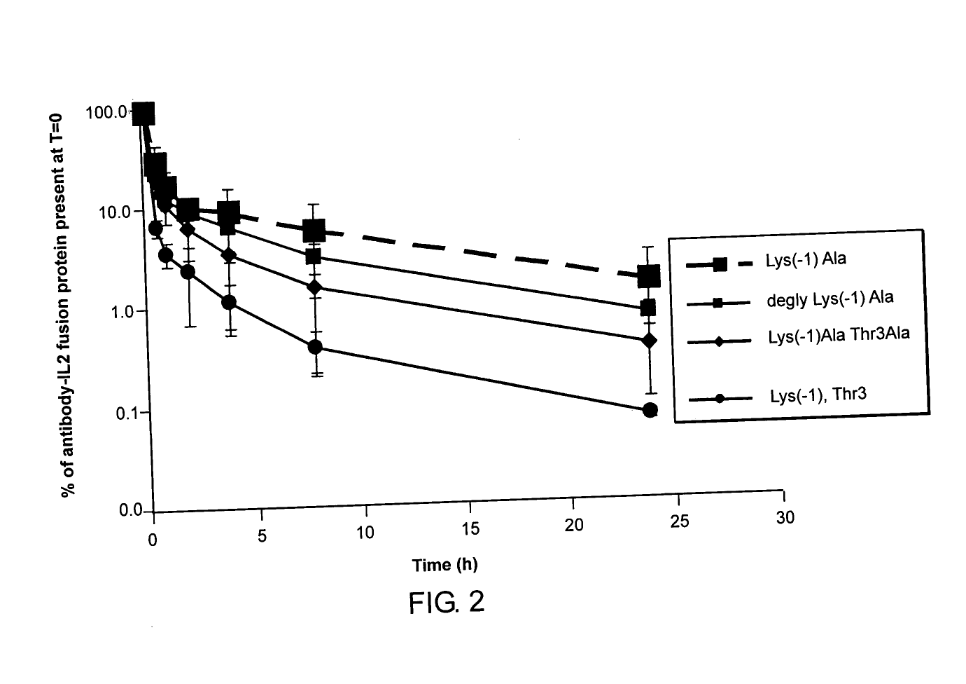

The effect of specific junctional alterations on the pharmacokinetics of antibody-IL-2 fusion proteins was investigated. Mice were injected with 25 micrograms of (i) antibody-IL-2 fusion protein containing no mutations (“Lys(-1) Thr3,” circles); (ii) fusion protein containing substitutions of the C-terminal lysine in the antibody heavy chain and at threonine 3 in IL-2 to alanines (“Lys(-1)Ala Thr3Ala,” diamonds); enzymatically deglycosylated protein with Lys(-1 )Ala (small squares); and a mutation of only the C-terminal lysine in the antibody heavy chain (large squares and dashed line). Blood samples were withdrawn at various times after injection and levels of intact fusion protein were measured. Results are shown in FIG. 2.

### Example 2

### Measurement of the Extent of O-glycosylation of IL-2 Fusion Proteins

In this example, the extent of O-glycosylation on the serum half-life of the IL-2 fusion protein was explored.

The extent of O-glycosylation in an IL-2 fusion protein was measured as follows. Antibody-IL-2 fusion proteins were expressed from genetically engineered mammalian NS/0 cells using standard procedures. The proteins were purified using Staph A protein according to standard techniques. The resulting purified antibody-IL-2 fusion proteins were analyzed by ion-exchange chromatography, and a distribution of peaks was observed using UV absorption. At the same time, a sample of the antibody-IL-2 fusion protein was treated with Sialidase (Roche Diagnostics GMBH, Mannheim Germany), and then analyzed using the same ion-exchange chromatography system (Agilent 1100 HPLC using a Dionex ProPac WCX-10 4.6 mm×250 mm column).

The reasoning behind this procedure was as follows. Sialidase removes terminal sialic acids from O-linked and N-linked glycans. In an antibody-IL-2 fusion protein, the only likely source of sialic acid is through O-glycosylation at threonine at position 3 of IL-2. There are no other known O-linked glycosylation sites in the antibody or IL-2.

A comparison of two ion exchange profiles showed that the untreated antibody-IL-2 fusion protein sample is distributed among up to five peaks, corresponding to 0, 1, 2, 3, or 4 sialic acid residues. After treatment with sialidase, the same material migrated as essentially a single peak. A comparison of the elution patterns provided an indication of the extent of O-glycosylation in the purified protein sample.

In one particular case, the extent of O-glycosylation in KS-Lys(-1)-IL-2, KS-Lys(-1)Ala-IL-2, and KS-Lys(-1)Ala-IL-2(Thr3Ala) were compared by the above method. The results indicated that KS-Lys(-1)-IL-2 was less than 5% O-glycosylated, KS-Lys(-1)Ala-IL-2 was at least 90% O-glycosylated, and KS-Lys(-1)Ala-IL-2(Thr3Ala) was not glycosylated at all. These results were confirmed by peptide mapping, based on tryptic digestion of the proteins and analysis of the resulting peptides by mass spectroscopy.

### Example 3

### Measurement of IL-2 Activity

This example was performed to determine the activity of the IL-2 fusion proteins. The activity of the antibody-IL2 fusion proteins was tested in four different cell-based assays.

For cell based bioassays, cell lines that depend on IL-2 for growth were utilized and the activity of Ig-fusion proteins, for example huKS-IL2 and huKS-IL2 variants, was assessed by proliferation of these cells. For instance, CTLL-2 (ATCC# TIB-214; Matesanz and Alcina, 1996) and TF-1β (Farner et al., [1995] Blood 86:4568-4578, the teachings of which are hereby incorporated by reference) were used to follow a T cell response and an NK cell-like response, respectively. CTLL-2 is a murine T lymphoblast cell line that expresses the high affinity IL-2Rαβγ, and TF-1β is a human cell line derived from immature precursor erythroid cells that express the intermediate affinity IL-2Rβγ. Another useful cell line for these assays is the cell line derived from human adult T cell lymphoma Kit-225 (K6) (Uchida et al., [1987] Blood 70:1069-1072, the teachings of which are hereby incorporated by reference). These assays were also performed with cell populations derived from human PBMCs (Peripheral Blood Mononuclear Cells) according to standard procedures.

As shown in Table 1 below the IL-2 activity of the antibody(Ala [-1])-IL-2(Thr3 Ala8 Ala9) fusion was essentially the same as for other antibody-IL-2 molecules.

TABLE 1

| | CTLL-2 | Kit-225 | TF-1B | PBMC IL2 |
|---|---|---|---|---|
| | ED50(ng/ | ED50(ng/ | ED50(ng/ | ED50(ng/ |
| Protein | ml) AVG | ml) AVG | ml) AVG | ml) AVG |
| KS ala IL2 | 1.86 | 0.09 | 0.46 | 1.63 |
| KS IL2 T3A | 1.13 | 0.04 | 0.89 | 1.70 |
| KS ala IL2 T3A | 1.33 | 0.07 | 1.14 | 1.89 |
| KS ala IL2 K8A, K9A | 1.91 | 0.05 | 1.78 | 2.40 |

### Example 4

### Characteristics of Albumin-IL-2 Fusion Proteins

This example is performed to investigate the serum half-life of the Albumin-IL-2 fusion protein (SEQ ID NO: 5) where threonine 3 of IL-2 is substituted with an alanine, i.e., an amino acid which cannot be O-glycosylated.

Albumin-IL-2 fusion proteins have a somewhat longer serum half-life than interleukin-2 alone. Albumin-IL-2 fusion proteins that are produced in eukaryotic cells such as mammalian cells or yeast are incompletely glycosylated at Threonine 3 of IL-2. Albumin-IL-2 fusion proteins in which the threonine 3 of IL-2 is substituted with alanine show less batch-to-batch variation than albumin-IL-2 fusion proteins in which threonine 3 of IL-2 is unaltered.

### Equivalents

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes which come within the meaning and range of equivalency of the claims are intended to be embraced therein.

### Incorporation by Reference

Each of the patent documents and scientific publications disclosed herein is incorporated by reference into this application in its entirety for all purposes.

1. A protein comprising an interleukin-2 protein, wherein Lys₈ and Lys₉ of the interleukin-2 protein are replaced with non-lysine amino acids.

2. A fusion protein comprising the protein of claim 1 and a carrier protein.

3. The fusion protein of claim 2, wherein the carrier protein is fused to the N-terminal portion of the interleukin-2 protein.

4. The protein of claim 1, wherein the non-lysine amino acids are hydrophobic amino acids.

5. The protein of claim 4, wherein the hydrophobic amino acids are selected from the group consisting of tryptophan, phenylalanine, tyrosine, methionine, glycine, alanine, leucine, isoleucine and valine.

6. The protein of claim 1, wherein the non-lysine amino acids are alanines.

7. The protein of claim 1, wherein the interleukin-2 protein is derived from a mammalian interleukin-2.

8. The protein of claim 1, wherein the interleukin-2 protein is derived from a human interleukin-2.

9. The protein of claim 3, wherein the N-terminal portion of the interleukin-2 protein comprises an O-glycosylation site.

10. The fusion protein of claim 2, wherein the carrier protein comprises albumin.

11. The fusion protein of claim 2, wherein the carrier protein comprises an immunoglobulin (Ig) moiety.

12. The fusion protein of claim 11, wherein the Ig moiety comprises at least a portion of an Ig heavy chain.

13. The fusion protein of claim 12, wherein at least one amino acid of the C-terminal portion of the Ig moiety is replaced with a hydrophobic amino acid.

14. The fusion protein of claim 13, wherein the C-terminal lysine residue of the Ig moiety is replaced with an alanine.

15. The fusion protein of claim 11, wherein the Ig moiety comprises at least the CH2 domain of an IgG2 or an IgG4 constant region.

16. The fusion protein of claim 11, wherein the Ig moiety comprises at least a portion of an IgG1 constant region where one or more amino acids selected from the group consisting of Leu₂₃₄, Leu₂₃₅, Gly₂₃₆, Gly₂₃₇, Asn₂₉₇, and Pro₃₃₁ are mutated or deleted.

17. The fusion protein of claim 11, wherein the Ig moiety comprises at least a portion of an IgG3 constant region where one or more amino acids selected from the group consisting of Leu₂₈₁, Leu₂₈₂, Gly₂₈₃, Gly₂₈₄, Asn₃₄₄, and Pro₃₇₈ are mutated or deleted.

18. The fusion protein of claim 2, further comprising a linker peptide between the carrier protein and the interleukin-2 protein.

19. A nucleic acid molecule encoding the protein of claim 1.

20. An expression vector containing the nucleic acid molecule of claim 19.

21. A cell comprising the nucleic acid of claim 19.

22. A process for preparing a protein comprising maintaining the cell of claim 21 under conditions permitting expression of the protein and harvesting the expressed protein.

23. A pharmaceutical composition comprising the protein of claim 1 and a pharmaceutically acceptable carrier.

24. A method of treating a disease in a mammal, the method comprising the step of administering to the mammal the composition of claim 23.

25. The method of claim 24, wherein the mammal is a human.

26. The method of claim 24, wherein the disease is selected from the group consisting of cancer, viral infection, and an immune disorder.

27. A method of treating a patient, the method comprising administering to the patient the nucleic acid of claim 19.

28. A method of treating a patient, the method comprising administering to the patient the cell of claim 21.

29. A method of determining the extent of O-glycosylation of an immunocytokine comprising:
providing an immunocytokine which has an O-glycosylation site;
measuring the level of O-glycosylation; and
comparing the level of O-glycosylation with a control.

30. The method of claim 29, wherein the immunocytokine comprises an immunoglobulin (Ig) and an interleukin-2 fusion protein.

31. The method of claim 30, comprising measuring the extent of O-glycosylation at Thr₃ of interleukin-2.
